(19) United States
(12) Patent Application Publication
(10) Pub. No.: US 2008/0033463 A1
(43) Pub. Date: Feb. 7, 2008

(54) Coated surgical blade

(76) Inventor: Drew Joseph Stoken, Carlisle, PA (US)

Correspondence Address: BINGHAM MCCUTCHEN LLP, 2020 K Street, N.W., Intellectual Property Department, WASHINGTON, DC, 20006, US

(21) Appl. No.: 11/498,853

(22) Filed: Aug. 4, 2006

(51) Int. Cl. A61B 17/32

(52) U.S. Cl. 606/167

(57) ABSTRACT

A method and apparatus for identifying an insertion area of a surgical blade is disclosed. It is desirable for an indicator to be applied to the surface of a surgical blade prior to being packaged for distribution, preferably during the manufacturing process. The indicator, which may include any tissue dye approved for surgical use, may be applied without damaging the cutting edge of the surgical blade. When the blade is inserted into a tissue, a portion of the indicator is transferred from the surface of the blade, or any other part of the device, to the tissue. After a surgeon performs the necessary incision and removes the blade, the indicator that has been transferred to the tissue aids in visually identifying the incision area. In this manner, subsequent manipulation of the incision area during the surgical process is facilitated.

### FIELD OF THE INVENTION

The present invention relates to surgical blades used during surgery. More specifically, the present invention relates to a dye coated surgical blade that visually identifies an area of incision.

### BACKGROUND OF THE INVENTION

The field of medicine has evolved over the centuries to include the ability to diagnose and treat various ailments. Modem medicine began by diagnosing a patient's ailments through observing a patient and their symptoms. As the field evolved, doctors have been able to perform more invasive procedures and tests, such as blood tests, magnetic resonance imaging (MRI's), and surgery. Most, if not all, invasive procedures have at least one characteristic in common; they require an incision into a patient's tissue.

Incisions have been made into tissues utilizing sharpened metal blades or diamond blades for several decades. One of the more recent advances in medicine is the ability to perform surgical procedures using smaller incisions. With respect to eye surgery in particular, smaller incisions allow for faster recuperation times and reduced pain, scarring, chance of infection, and distortion of the wound, allowing less induced astigmatism. One of the difficulties that arise when performing small incisions in eye tissue is that the incision can become invisible even under the magnification of an operating microscope. This leads to attempts to identify the incision through further manipulations, causing increased trauma, distortion of the wound, increased surgical time, and postoperative pain.

One method currently being used is to mark the knife blade with a “skin scribe,” which is a sterile marking pen filled with an indicator, such as a suitable or approved tissue dye. Unfortunately, this method has many limitations. Primarily, it requires purchasing an individual marker for each case, thereby increasing the cost per case. It also takes some time for the surgeon or assistant to apply the dye, leading to increased operating time. In particular, the surgeon has to specifically remember to apply the dye so that it is not inadvertently omitted. Moreover, the pen that is used to mark the incision needs to be remembered, e.g., the surgeon does not want to leave the pen inside the body. This is especially important, for example, when performing a surgery that requires a large incision, e.g., in the thoracic or abdominal region, allowing for the possibility of a surgeon accidentally forgetting the marking pen in the human tissue. Damage to the cutting edge of the blade may also potentially occur, leading to more difficult or traumatic surgery. To further complicate matters, the skin scribe can result in inconsistent application of the dye to the blade, e.g., too much or too little dye, which in turn results in inconsistent application to the incision.

Therefore, a continuing need exists for a method and apparatus that is capable of identifying an incision without increasing the surgical procedure time or trauma to a patient.

### SUMMARY OF THE INVENTION

According to one embodiment, the present invention comprises an apparatus for identifying an insertion area. The apparatus includes an insertion device comprising at least one sharpened edge and an indicator applied to at least part of the surface of the insertion device. Preferably, the indicator is applied to the surface such that at least some of the indicator is transferred from the surface of the insertion device to a tissue when the surface contacts the tissue. Optionally, the insertion device may also include at least one of a shaft or a handle to aid a medical professional in manipulating the sharpened edge.

In one embodiment, the indicator comprises a tissue dye approved by the Food and Drug Administration for use during surgical procedures. Examples of approved dyes include, but are not limited to, Gentian violet, Lissamine green, Indocyanine Green, or Trypan blue. The dye may be applied to about 75% or more of the surface of the sharpened edge. When the sharpened edge is inserted into a tissue, at least some of the indicator is transferred to the tissue, thereby visually identifying the insertion area.

According to another aspect, the present invention comprises a method for identifying an insertion area in a tissue. The method includes providing an insertion device that includes a substance that is applied to a portion of the surface of the insertion device. Then, the device is inserted into the tissue. During the insertion process, at least a portion of the substance on the surface of the insertion device is transferred to the tissue. The insertion device may then be removed from the tissue. As a result, the substance transferred to the tissue indicates an insertion area of the insertion device. In some embodiments, at least a portion of the substance on the insertion device may be transferred to the tissue during the process of removing the insertion device.

The substance may be applied to a portion of the surface of the insertion device in a variety of manners. For instance, in one embodiment the substance may be applied to the insertion device using a holding and dispensing device that includes the substance, e.g., an indicator. Regardless of the method or apparatus used, the substance is preferably applied without damaging the cutting edge of the insertion device.

According to yet another aspect of the present invention, a method of applying an indicator to a blade is disclosed. The method includes manufacturing a blade having a cutting edge and applying the indicator onto at least a portion of the blade. It is desirable for the applying to be performed without substantially damaging the cutting edge of the surgical blade. Preferably, the indicator is applied to a portion of the blade prior to packaging the blade for distribution, e.g., during the manufacturing process.

In one embodiment, the indicator may be applied to the blade using a spray or brush. Alternately, the indicator may be applied using a holding and dispensing device comprising the substance, such as a pen and the like. In some embodiments, the blade may be operatively connected to a shaft or handle, both of which may also have the indicator applied thereon. After the indicator is applied to the blade, the blade may be inserted into a tissue. During the time when the blade is inserted into the tissue, at least some of the indicator is preferably transferred from the surface of the blade onto the tissue. After a desired incision has been made, the blade may be removed from the tissue.

The present invention also includes a method for identifying an insertion area in a tissue. The method includes providing a blade having a cutting edge. An indicator is preferably applied to a portion of the surface of the blade in a substantially uniform manner without damaging the cutting edge of the blade. After the indicator is applied, the blade is placed in sterile packaging for distribution. When desirable, a medical professional may remove the blade from the sterile packaging and use it to make an incision.

### BRIEF DESCRIPTION OF THE DRAWINGS

FIG. 1 shows an exemplary blade according to one aspect of the present invention;

FIG. 2 shows an exemplary blade, shaft, and handle according to one aspect of the present invention;

FIG. 3 shows an exemplary keratome according to one aspect of the present invention;

FIG. 4 shows the exemplary keratome of FIG. 3 penetrating eye tissue according to one aspect of the present invention; and

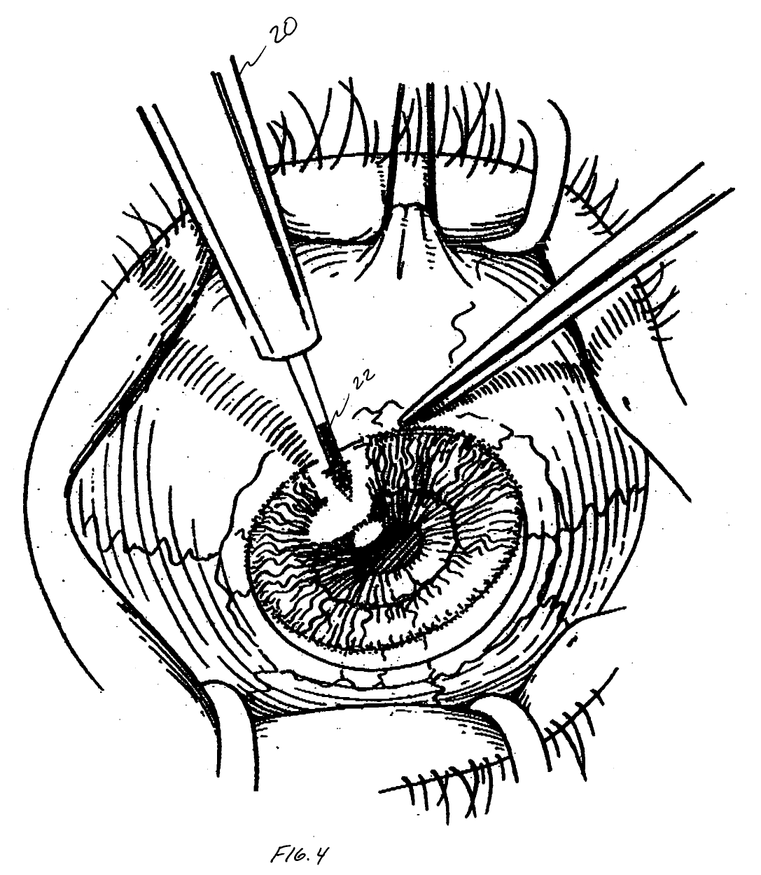

FIG. 5 shows a marked insertion point according to one aspect of the present invention.

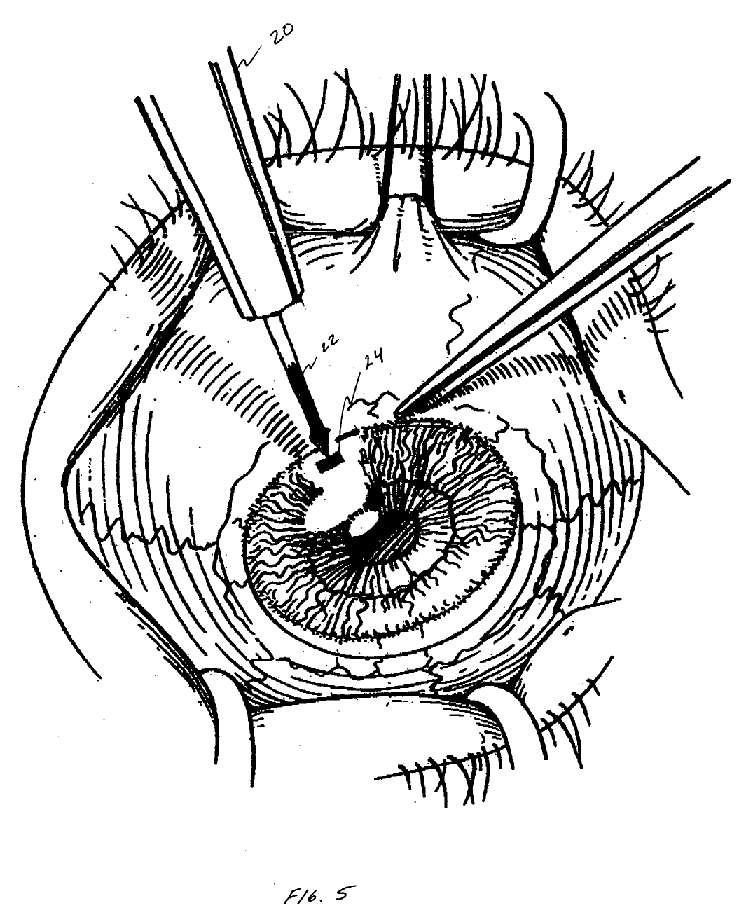

### DETAILED DESCRIPTION OF THE PREFERRED EMBODIMENTS

In the process of diagnosing and treating illnesses, diseases, and other health or medical concerns, or performing elective surgeries, medical professionals often make incisions into living tissue. Depending on the particular type of surgery, the incision may be used to alter a part of the body, e.g., by inserting or removing a foreign body or substance, or to further inspect or otherwise alter a particular part of the body. While there are a variety of options available to a medical professional for making incisions, a sharpened edge, such as a blade, knife, keratome, or other object that is capable of exerting high pressure over a small surface area is typically employed.

Many living creatures, such as human beings, have a variety of different types of tissues, e.g., muscle tissue, fatty tissue, brain tissue, and the like. The various types of tissues, however, may generally be grouped into two categories: vascular tissue and avascular tissue. As skilled artisans will appreciate, vascular tissue is tissue that contains blood vessels, while avascular tissue does not have any blood vessels. Due to the absence of blood vessels, avascular tissue appears clear to the naked human eye, making it difficult for a surgeon to visually identify an area of incision after a blade has been removed. This is the case, for example, when performing surgery on the eye, where the avascular tissue makes it difficult for the unaided human eye to see where an incision has been made after a blade has been removed. In particular, incisions into the cornea and the avascular sclera may be particularly difficult to identify.

In order to assist a surgeon or other medical professional that is making an incision in tissue, e.g., eye tissue, the present invention provides a method and apparatus that marks the area where a blade has been inserted, e.g., an insertion area, during the incision process. One advantage of the present invention is that it eliminates the need to use other methods of identifying an incision that can be unreliable or inaccurate, reducing the trauma to a patient's body. According to one aspect, the present invention includes a sharpened edge that includes a substance, e.g., an indicator, that visually identifies the insertion area of the sharpened edge during the incision process. In one embodiment, the substance may be detectable by the human eye, i.e., it imparts a color within the visual spectrum to the incision area. In other embodiments, it may be desirable for the substance to be visually identifiable through the use of special equipment, such as special lenses, filters, and or illumination sources.

As mentioned above, the indicator is preferably applied to the surface of a sharpened edge, such as a keratome. The surface of the sharpened edge may be altered in a variety of desired manners. For example, the surface of the sharpened edge may be modified to include “dimples” or “grooves,” or generally roughened to provide an area for the indicator to be deposited, thereby facilitating the coalescing or drying of the indicator. In other embodiments, the indicator may also be deposited on any other surface that is attached to the blade including, but not limited to, the unsharpened handle and/or shaft, described in more detail below. If and/or when the present invention is inserted further into an tissue (by way of the incision), contact with the shaft and/or unsharpened handle would provide the advantage of applying the indicator to parts of the tissue beyond the incision area. It is desirable for the indicator to be applied to the surfaces in a substantially uniform manner. The uniform application of the indicator to the surface of sharpened edge increases the chances of the dye being transferred to the tissue uniformly.

Once the indicator dries and adheres to the surface of the blade, it may undergo subsequent processing, e.g., attaching a handle to the blade before being packaged and shipped to various locations. The packaging is preferably sterile packaging that prevents bacteria and other infectious agents from contaminating the blade. To prevent the blade from causing an infection when it is used, e.g., due to bacteria, viruses, and the like, it is preferably sterilized before the indicator is applied to its surface. Optionally, the blade may be sterilized again after the indicator has dried. Preferably, the indicator is fast drying, i.e., it dries soon after it is applied, in order to allow the blade to undergo subsequent manufacturing processes, e.g., packaging and the like.

Preferably, the indicator dries in about 1 minute or less after it is applied to the surface of the blade. More preferably, the indicator dries about 45 seconds or less after it is applied to the surface of the blade. Most preferably, the indicator dries about 30 seconds or less after it is applied to the surface of the blade. In another embodiment, the indicator preferably dries between about 1 and about 60 seconds after it is applied to the surface of the blade. More preferably, the indicator dries between about 1 and about 30 seconds after it is applied to the surface of the blade. Most preferably, the indicator dries between about 1 and about 15 seconds after it is applied to the surface of the blade.

In one embodiment, it is desirable for the indicator to comprise a dye. The dye that is used may comprise any color within the visual spectrum, such as red, orange, yellow, green, blue, indigo, violet, or any combination thereof. Of course, it is desirable for the dye to be safe for use in the human body during surgical procedures. Examples of dyes that may be used in accordance with the present invention include Gentian violet, Lissamine green, Trypan blue, Indocyanine green, or any other tissue dyes approved for use during surgical procedures by the Food and Drug Administration. Moreover, other compatible tissue dyes known to those skilled in the art may be used. Alternately, the indicator may comprise a fluorescent dye that can be excited by light having a predetermined wavelength. In other embodiments, the indicator may not be visible to the naked human eye. Examples of such substances include, but are not limited to, a dye that responds to light within the ultraviolet (UV) spectrum.

In embodiments where a fluorescent dye is used, it may be desirable to excite the dye using an illumination source that generates light within a predetermined, limited spectrum. To increase the ability of the surgeon to see the fluorescent dye, a medical professional may use glasses having filtered lenses that prevent light outside the emission wavelength from passing through to his/her eye. In alternate embodiments that employ a UV indicator, it may be preferable to use an illumination source that generates light within the UV spectrum.

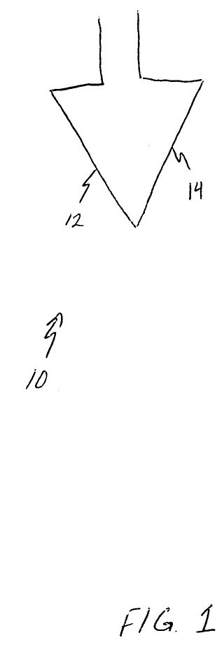

With reference to FIG. 1, an exemplary blade according to one aspect of the present invention is described. As mentioned above and shown in FIG. 1, the present invention includes a sharpened edge 10, such as a knife, blade, or keratome. The knife or blade 10 may be comprised of any substance known to those skilled in the art, including metal, plastic, alloys, composite, glass, diamonds, or combinations thereof. The blade 10 may have any desired dimensions, such as size, shape, or thickness. In one embodiment, the blade 10 comprises a keratome that includes two sharpened edges 12, 14. Though the blade is shown and described having two sharpened edges 12 and 14, the blade 10 may have more than two, or only one, sharpened edge. The present invention is not intended to be limited to any particular type of sharpened edge or cutting device. Skilled artisans will recognize that any sharpened edge or cutting device known to those skilled in the art may be used in accordance with the present invention.

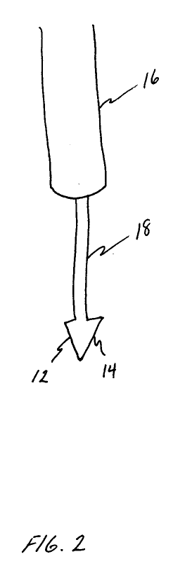

As shown in FIG. 2, the blade is preferably attached to a handle 16 using a shaft 18. The handle 16 and the shaft 18 may comprise any material known to those skilled in the art including, but not limited to, plastic, metal, alloys, composite, glass, or combinations thereof. Moreover, the handle 16 and shaft 18 may have any desired dimensions, such as size, shape, or thickness. In particular, it may be desirable for the handle 16 to be configured and dimensioned to allow a medical professional to easily manipulate the device.

According to the present invention, the indicator may be applied to the surface of the blade in a variety of manners. In one exemplary embodiment, it may be desirable for the indicator to be maintained in a liquid state to facilitate transport and/or storage. When it is desirable, the liquid indicator may be applied to the surface of the blade by converting it to a spray or fine mist using a nebulizer or other type of device known to those skilled in the art. According to another aspect, the indicator may be applied to the surface of the blade using a roller or brush. As such, the indicator may be applied to the roller or brush, which may then be used to transfer the ink to the surface of the blade. Alternately, the indicator may be applied to the surface of the blade by dipping it into the indicator. The indicator may also be manually or mechanically applied by using an indicator filled “pen,” or similar holding and/or dispensing device. Those skilled in the art will understand that the present invention is not intended to be limited to any particular method of application described herein. Along those lines, the methods described herein are for illustrative purposes only, and may be modified in any desirable manner, consistent with the instant teachings, by those skilled in the art. Regardless of the method or apparatus employed to apply the indicator to the insertion device, it is desirable that it is applied in a manner that does not damage the cutting edge of the knife blade. Further, the indicator may be applied to surfaces other than the blade, such as the shaft 18 shown in FIG. 2.

It may be desirable to apply the indicator to only a predetermined portion of the blade to assist a surgeon in making incisions, as described in detail below. For instance, it may be preferable to apply the indicator to about 25% or more of the surface area of the blade. More preferably, it may be desirable to apply the indicator to about 50% or more of the blade. Most preferably, it is desirable to apply the indicator to about 75% or more of the blade. Another way to describe the portion of the blade to which the indicator is applied is by the ratio of the part of the blade that has indicator applied to the part of the blade to which the indicator is not applied. In this embodiment, the ratio of the part of the surface covered with the indicator to the part of the surface that is not covered is about 1:3 or greater. More preferably, the ratio of the part of the surface covered with the indicator to the part of the surface not covered by the indicator is about 1:1 or greater. Most preferably, the ratio of the part of the surface covered with the indicator to the part of the surface that is not covered is about 3:1 or greater.

In some applications, it may be desirable for the ink to be applied to a portion of the blade above or below a certain point on the blade, e.g., the top part of a blade. This may be desirable, for example, in order to assist a surgeon in determining when the blade has been inserted deep enough into a tissue. For instance, it may be desirable to generate an incision in the eye that is about 0.2 mm deep. In order to assist the surgeon in making an incision that does not go deeper than about 0.2 mm, the indicator may be applied to the portion of the blade that is about 0.2 mm from the tip of the blade. Thus, when the blade has been inserted into the eye about 0.2 mm or greater, the indicator will then be transferred to the eye tissue. When the incision is performed slowly, the blade thereby assists the physician in making the incision of the proper depth, i.e., the physician knows that the incision is deep enough when the ink begins to be transferred to the eye tissue.

A similar principle may be used in the opposite manner. Continuing with the example provided above, it may be desirable for a surgeon to perform an incision that is about 0.2 mm deep. That is, ink may be applied beginning at the tip of the blade and proceeding up to about 0.2 mm. Then, when a surgeon inserts the blade to make an incision, the ink will be transferred to the eye tissue. When the portion of the blade comprising the indicator is fully inserted into the eye tissue, the surgeon will know that an incision of sufficient depth has been made. Skilled artisans will recognize that the indicator may be applied to the bottom portion of the blade in any desirable manner, e.g., by limiting the portion of the blade dipped into the ink, by applying the ink to the desired portion using a roller or brush, or by covering the top portion of the blade and spraying ink onto the blade.

According to one aspect of the present invention, the entirety of the indicator that is present on the surface of the blade may not be transferred to the tissue. In other words, only a portion of the indicator included on the surface of the blade may be transferred to the tissue when an incision is made. However, the amount of the indicator that is transferred to the tissue is preferably sufficient to identify the incision area.

The indicator that is transferred to the tissue preferably remains in the tissue for a predetermined period of time that is sufficient to allow the medical professional to perform further procedures near, inside, or around the insertion or incision area. After the predetermined period of time expires, however, it is desirable for the ink to be capable of dissolving or otherwise disseminating safely within the human body. Preferably, the indicator is capable of identifying the insertion area for about 30 minutes or more. More preferably, the indicator is capable of identifying the insertion area for about 45 minutes or more. Most preferably, the indicator is capable of identifying the insertion area for about 60 minutes or more.

The use of indicators such as fluorescent or UV indicators may require special equipment in order to be detected by the human eye. Consider, for example, the use of a fluorescent indicator that is applied to the surface of the blade. When the blade is used to make an incision, at least some of the fluorescent indicator is preferably transferred to the eye tissue, as described above. In order to assist the medical professional in identifying the insertion area, the surgeon may wear eye glasses that include filters that substantially prevent light outside the emission spectrum of the fluorescent indicator from passing to the surgeons eye. In this embodiment, a surgeon may wear the glasses only while performing certain procedures, such as making or manipulating the incision. If desired, however, the surgeon may wear the glasses while performing the entire surgery. A limited spectrum light source, i.e., a light source that is limited to producing the excitation wavelength for a fluorescent indicator, may be used to excite the fluorescent indicator. The limited spectrum light source may include a filter that is part of, or operatively connected to the light source. Alternately, a light source that provides a broad spectrum of light, e.g., white light, may be used. Or, the light source may comprise one or more light emitting diodes (LED's) that produce light within either a limited or broad spectrum.

According to an exemplary method of the present invention, a medical device manufacturer applies an indicator to the surface of a medical device, e.g., a knife, keratome, catheter, and/or intravenous device. Preferably, the application of the indicator is part of the manufacturing process, although it may be performed independently if desired. The manufacturing process of the medical device may include a variety of steps prior or subsequent to the application of the indicator. Examples of these steps include, for example, connecting a handle to a blade, sterilization, sharpening, and the like.

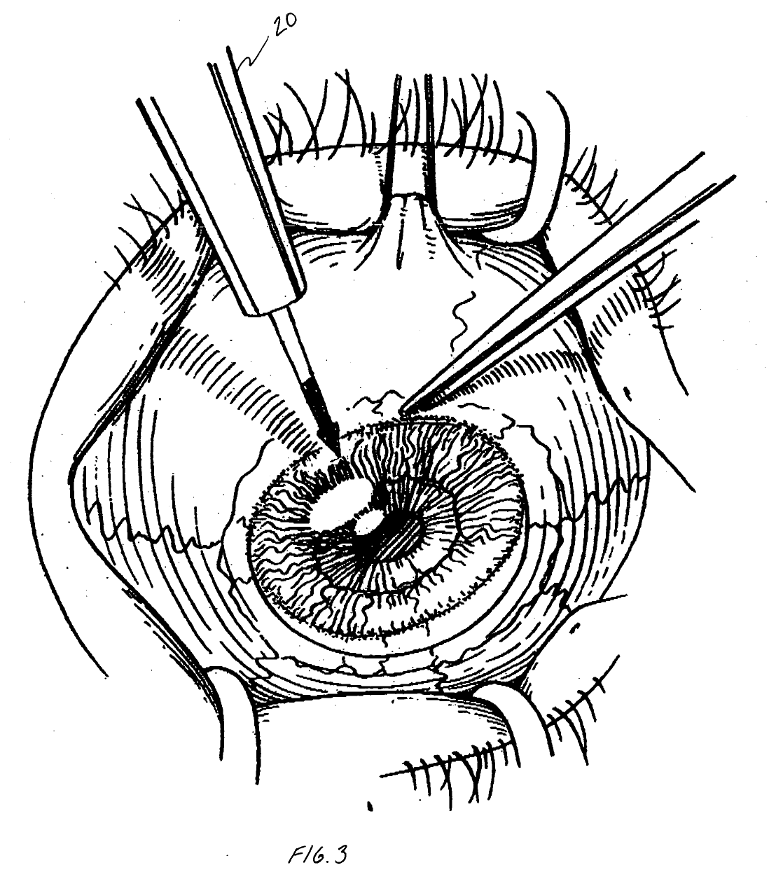

One example of a keratome manufactured according to the present invention is shown in FIG. 3. As shown in FIG. 3, when performing a medical procedure, a medical professional may obtain the keratome 20 and determine where an incision is to be made. After the appropriate area has been identified by the surgeon, the keratome 20 may be inserted into the eye, generating the incision. As the keratome 20 passes through the eye tissue, at least some of the indicator 22 is transferred from its surface to the eye tissue. Once an appropriate incision has been made, the keratome 20 may be withdrawn from the tissue, thereby allowing the surgeon to visually identify the incision 24. Thus, the apparatus and method of the present invention facilitate subsequent manipulation of the incision 24, minimizing human error, causing less trauma to a patient, and reducing the time necessary for a medical procedure to be performed.

The present invention is not limited to use with sharpened edges. Other insertion devices used by medical devices may have an indicator applied to its surface, including tubes, stents, catheters, and the like. Moreover, the present invention is capable of being used with any type of tissue, whether vascular or avascular. In addition, the invention described herein may be used with humans and other animals, such as cats, dogs, mice, rats, birds, and the like. As mentioned above, the present invention may be used during surgery on the eye, such as cataract surgery, retinal surgery, glaucoma surgery, corneal surgery. The present invention is particularly useful when used for incision placed into the clear cornea, corneal limbus, conjunctiva, and sclera. While the present invention is described with respect to surgery performed on the eye, it is not intended to be limited to use with any particular part of the body. In other embodiments, the present invention may be used during procedures that are performed in all other surgical specialties, including orthopedics, neurosurgery, vascular surgery, urologic surgery, otolaryngologic surgery, plastic/reconstructive surgery, cardiovascular surgery, podiatric surgery, general surgery, and gynecologic surgery.

Although the present invention has been described with reference to particular embodiments, it will be understood to those skilled in the art that the invention is capable of a variety of alternative embodiments within the spirit of the appended claims. For example, according to another aspect of the present invention, the blade of the present invention may be include a reservoir or cavity that is capable of holding a substance, such as the indicator described above. As such, the blade may be impregnated with the substance, rather than having the substance applied to portions of its outer surface.

In one embodiment, the blade preferably includes one or more apertures through which the substance may pass to the outside surface of the blade. Moreover, the size of the apertures may be increased or decreased to selectively prevent or allow the substance within the reservoir to pass to the outside of the blade. In other words, when the aperture is restricted, the substance is substantially prevented from passing to the outside of the blade. Conversely, when the aperture is opened, the substance is free to pass to the outside surface of the blade. When a blade is inserted into a tissue and the aperture is open, or opened subsequent to insertion into the tissue, the substance may be transferred to the tissue as a result, thereby visually identifying the insertion area.

To facilitate the opening and/or restriction of the one or more apertures, the blade may include a shaft and/or a handle that is capable of interacting with the blade to open or restrict the apertures. In other embodiments, the shaft and/or handle may include reservoirs or cavities of their own. One advantage of this embodiment is that the substance may be inserted into the handle's reservoir. Thereafter, the substance may pass through the cavity of the shaft and settle into the blade's reservoir, which is operatively connected to the handle's reservoir. In such an embodiment, it may be desirable for the handle and/or shaft to include an actuator, e.g., a button, that allows a medical professional to easily open or close the blade's apertures.

Heretofore, the present invention has been described with respect to use by a medical professional. Increasingly, however, many aspects of the medical profession have benefited from the use of machines, particularly machines that include processors, e.g., computers. As such, the present invention may also be used in combination with a machine that manipulates the blade. The machine may be controlled by a medical professional, or more preferably it may be controlled by a computing device. In such an embodiment, a computer controlled machine may be programmed to make an incision in a particular tissue. The present invention, manipulated by the machine, may automatically generate an incision under the medical professional's supervision.

Moreover, though the present invention has been described with respect to a sharpened edge, is it not intended to be limited to any type of medical equipment. In other words, the method of the present invention may be used in combination with any piece of equipment used by medical professionals to cut, hold, or otherwise manipulate tissue. In this manner, the contact area of medical equipment with a tissue may be visually identifiable to a medical professional for a desired amount of time. Thus, devices including, but not limited to probes, clamps, scissors, and picks, may be used according to the present invention as described herein.

1. An apparatus for identifying an insertion area, comprising:
an insertion device comprising at least one sharpened edge; and
an indicator applied to at least part of a surface of the insertion device;
wherein the indicator is applied to the surface such that at least some of the indicator is transferred from the surface of the insertion device to a tissue when the surface contacts the tissue.

2. The apparatus according to claim 1, wherein the insertion device comprises at least one of a shaft or a handle.

3. The apparatus according to claim 1, wherein the indicator is applied to about 75% or more of the surface.

4. The apparatus according to claim 1, wherein the indicator comprises a tissue dye approved for use during surgical procedures by the Food and Drug Administration.

5. The apparatus according to claim 1, wherein the indicator transferred to the tissue visually identifies the insertion area.

6. The apparatus according to claim 4, wherein the tissue dye comprises at least one of Gentian violet, Lissamine green, Indocyanine Green, or Trypan blue.

7. A method for identifying an insertion area in a tissue, comprising:
providing a blade having a cutting edge, wherein an indicator is substantially uniformly applied to a portion of the surface of the blade;
placing the blade in sterile packaging for distribution after applying the indicator;
removing the blade having the indicator from the sterile packaging; and
generating an incision using the blade;
wherein the indicator is applied to a portion of the surface of the blade without substantially damaging the cutting edge of the blade.

8. The method according to claim 7, wherein the indicator is applied to the blade using a brush.

9. The method according to claim 7, wherein the indicator is capable of being transferred to a tissue by physical contact.

10. The method according to claim 7, wherein the indicator is applied to the blade using a spray.

11. The method according to claim 7, wherein the indicator comprises at least one of Gentian violet, Lissamine green, Indocyanine green, or Trypan blue.

12. The method according to claim 7, wherein the blade further comprises at least one of a handle or a shaft.

13. A method of applying an indicator to a blade, comprising:
providing a blade having a cutting edge; and
applying the indicator onto at least a portion of the blade, wherein the applying is performed without substantially damaging the cutting edge of the blade, and wherein the indicator is applied to a portion of the blade prior to packaging the blade for distribution.

14. The method according to claim 13, wherein the indicator is applied to the blade during the manufacturing process.

15. The method according to claim 13, wherein the indicator is applied to the blade using a spray.

16. The method according to claim 13, wherein the indicator is applied to the insertion device using a holding and dispensing device including the substance.

17. The method according to claim 13, wherein the indicator is applied to the blade using a brush.

18. The method according to claim 13, further comprising:
inserting the blade into a tissue;
transferring at least some of the indicator from the surface of the blade onto the tissue; and
removing the blade from the tissue.

19. The method according to claim 18, wherein the indicator transferred to the tissue visually identifies an insertion area of the blade.

20. The method according to claim 18, further comprising at least one of a handle or a shaft, wherein the indicator is applied to at least one of the handle or the shaft.
